(19) United States
(12) Patent Application Publication
(10) Pub. No.: US 2026/0157773 A1
(43) Pub. Date: Jun. 11, 2026

(54) BONE FRACTURE FIXATION DEVICE WITH RATCHETING CLAMP

(71) Applicant: Stabiliz Orthopaedics, Inc., Exton, PA (US)

(72) Inventors: Douglas Cerynik, Wynnewood, PA (US); Roy W. Sanders, Exton, PA (US); Sergio Gutierrez, Exton, PA (US); James Michelinie, Exton, PA (US); Chad DeRosier, Exton, PA (US)

(73) Assignee: Stabiliz Orthopaedics, Inc., Exton, PA (US)

(21) Appl. No.: 19/369,247

(22) Filed: Oct. 26, 2025

Related U.S. Application Data
(60) Provisional application No. 63/710,637, filed on Oct. 23, 2024.

(51) Int. Cl. A61B 17/64; A61B 17/00

(52) U.S. Cl. CPC A61B 17/6416; A61B 2017/00407

(57) ABSTRACT

A clamping device engages an external fixation rod for use in bone fracture surgery includes an upper housing including a ratcheting mechanism, a lower housing including an upper jaw and a lower jaw that engages an external fixation rod. When the upper housing is turned in a clockwise motion relative to the lower housing, the jaws are drawn towards one another until opposed by a predetermined force. Engaging an action pin in the upper housing and turning the upper housing in a clockwise motion can overcome the predetermined force, and engaging an action pin in the upper housing and turning the upper housing in a counterclockwise motion can release engagement to an external fixation rod.

### BACKGROUND

Bone fracture fixation technology can involve the insertion of pins or screws into the bone and manipulating the bones, through movement of the screws, to ensure proper bone alignment. To minimize exposing more tissue to infections, the screws may be inserted into the bone and manipulated from outside the skin barrier.

Once manipulated into a proper position, a surgeon may want to hold the screws and bone in place, and they may do this by connecting one or more of the screws to one another through a clamping system that connects the screws to one another through a series of connector rods clamps, and joints.

Some of the problems faced in using these clamping systems include:
overtightening of clamps to an unnecessary degree;
undertightening of clamps below a necessary threshold to maintain secure bone screw fixation;
material choices that require autoclave cleaning for re-use;
material choices and designs that are hard to adequately clean;
inability to engage both a fixation rod and fixation device like a screw or pin;
other problems.

The clamps and certain of the clamping system elements may be improved as shown herein.

### SUMMARY OF THE EMBODIMENTS

This invention concerns fixation devices for the treatment of bone fractures. More specifically, the invention describes a clamping device and system that engages bone screws, clamps, and external fixation rods together.

A clamping device configured to engage an external fixation rod for use in bone fracture surgery includes an upper housing including a ratcheting mechanism, a lower housing including an upper jaw and a lower jaw that engages an external fixation rod. When the upper housing is turned in a clockwise motion relative to the lower housing, the jaws are drawn towards one another until opposed by a predetermined force. Engaging an action pin in the upper housing and turning the upper housing in a clockwise motion can overcome the predetermined force, and engaging an action pin in the upper housing and turning the upper housing in a counterclockwise motion can release engagement to an external fixation rod.

### BRIEF DESCRIPTION OF THE DRAWINGS

FIG. 1 shows an enlarged cross section of the clamping mechanism described herein.

FIG. 2 shows a further cross section of the view in FIG. 1.

FIGS. 3-6 show exploded views of the clamping mechanism.

FIG. 7 shows another cross sectional view through the clamping mechanism.

FIG. 8 shows an exterior perspective view of the clamp.

FIG. 9 shows an elevation view of the clamp.

FIG. 10 shows a top view of the clamp.

FIG. 11 shows a partial section view of the clamp.

FIGS. 12A-12F show partial section views of the clamp, with some parts shown transparent for clarity.

FIGS. 13A-13C show partial section views of the clamp, with some parts shown transparent for clarity.

FIG. 14 shows a cross section through the clamping mechanism.

FIG. 15 shows a close up view of the view of FIG. 14.

### DETAILED DESCRIPTION OF THE EMBODIMENTS

U.S. Pat. Nos. 9,597,117 and 11,213,334 describe different bone connector and alignment technologies and are incorporated by reference as if fully supported herein. These patents and others describe the general technologies used to manipulate bones and align them during surgeries.

The clamping system in its entirety includes at least bone fasteners 200 (pins or screws, more often referred to as screws herein) that a surgeon drives into a bone engaged to clamps 100 that are in turn engaged to external fixation rods 300.

As shown in FIG. 107, the bone screw 200 may have threading 210 at an end thereof that engages the bone. The bone screw 200 may also be delivered to its bone engagement site through a bone screw sheath 250 with a channel 260 therethrough that is shaped to receive the bone screw 200.

In use, a surgeon may slide the bone screw sheath 250 (with the bone screw 200 therein) into the clamp bone screw channel 110 using a handle 252 that may be removably engaged to the bone screw sheath in a press fit. The handle allows the surgeon to remove the bone screw sheath once the surgeon engages the bone screw 200 to the bone.

Once the surgeon engages the bone screws 250 to their sites, and slides the clamps 100 onto the cone screws 250 (and/or sheaths 252), the surgeon engages the clamps 100 to external fixation rods 300 to hold the bone screws in a position that best aids the healing process.

### The Clamp

The clamp 100 includes a lower clamp housing 120 and an upper clamp housing 150. Clockwise rotation of the upper clamp housing 150 relative to the lower clamp housing 120 (to a certain predetermined torque limit to be discussed below) draws the upper and lower clamp jaws 122, 124 towards one another. Continued rotation beyond the predetermined torque results in no further movement of the jaws toward one another, unless the surgeon depresses the action pin 106, and continues the clockwise movement of the upper clamp housing 150 relative to the lower clamp housing 120, effectively overriding the predetermined torque limit.

If the surgeon moves the upper clamp housing 150 counterclockwise with respect to the lower clamp housing 120, the clamp jaws do not move. If the surgeon depresses the action pin 106 and then rotates the upper clamp housing 150 counterclockwise with respect to the lower clamp housing 120, then the jaws separate for manipulation of the clamp 100 with respect to the external fixation rod 300 or with respect to the bone screw 200.

### Clamp Mechanism

The lower clamp housing 120 and upper clamp housing 150 rotate with respect to one another about a hollow central axle 126. The hollow central axle includes a threaded portion 126a engaged to components in the upper clamp housing 150, and a larger split bulbous end 126b that engages the lower jaw 124, which is inside the lower clamp housing 120.

The upper clamp housing 150 includes two pieces shaped to engage one another: an upper piece 150a and a lower piece 150b. The two pieces form between them a channel 150a1, 150a2 that holds the action pin 106 and its associated outward biasing spring 106a.

The drive screw 140 has internal threads 142 that engage the central axle 126's external threads 126a, such that rotation of the drive screw 140 in a clockwise direction draws the central axle 126 further into the upper clamp housing 150, and counterclockwise does the reverse. The central axle's movement closes and opens the jaws, 124 respectively, due to the split bulbous end 126b's engagement with the lower jaw 126 and downward pressure of the toothed pressure plate 191 against the top of the upper jaw 122.

The drive screw 140 has an outer threading 144 at one end that engages a drive screw nut 148, which holds a wave spring washer 147 and a slip cam washer 160 in place against the drive screw gearing 143. The drive screw gearing 143 engages the action pin 106 when it is depressed, and when depressed, rotation of the upper housing 150a drives the drive screw 140. An outer drive washer 170 engages the slip cam washer 160 in such a way that any rotation of one, rotates the other. Finally, an inwardly biased ratchet pin 108 (with an associated spring 108a) engages this outer drive washer 170.

The figures show several wear washers not described herein that may be used to reduce wear between components, especially at friction interfaces, and given there may be a mix of thermoplastic and metal components.

### The Clamp in Use

### Tightening to a Predefined Torque

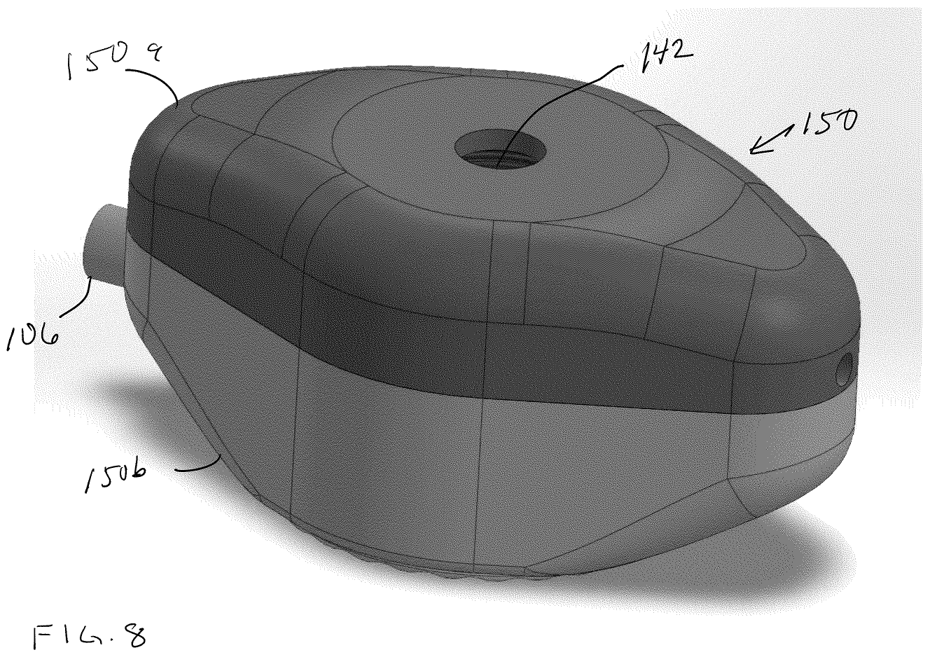

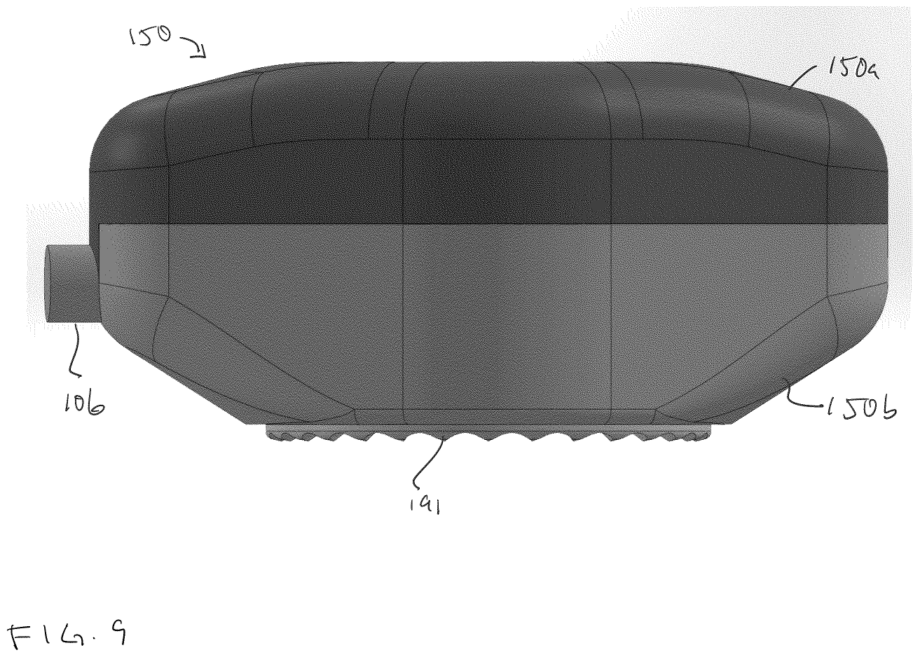

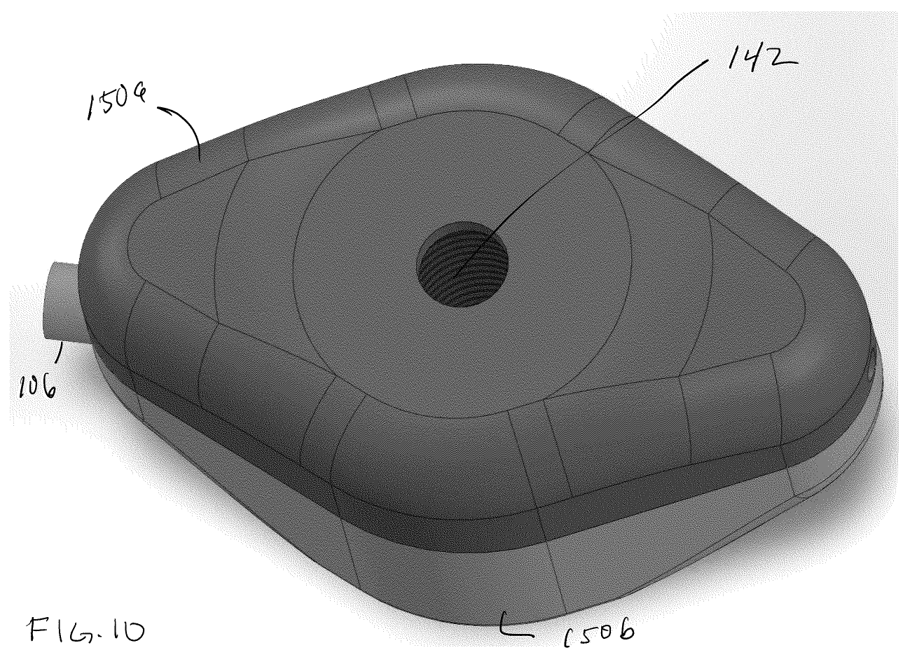

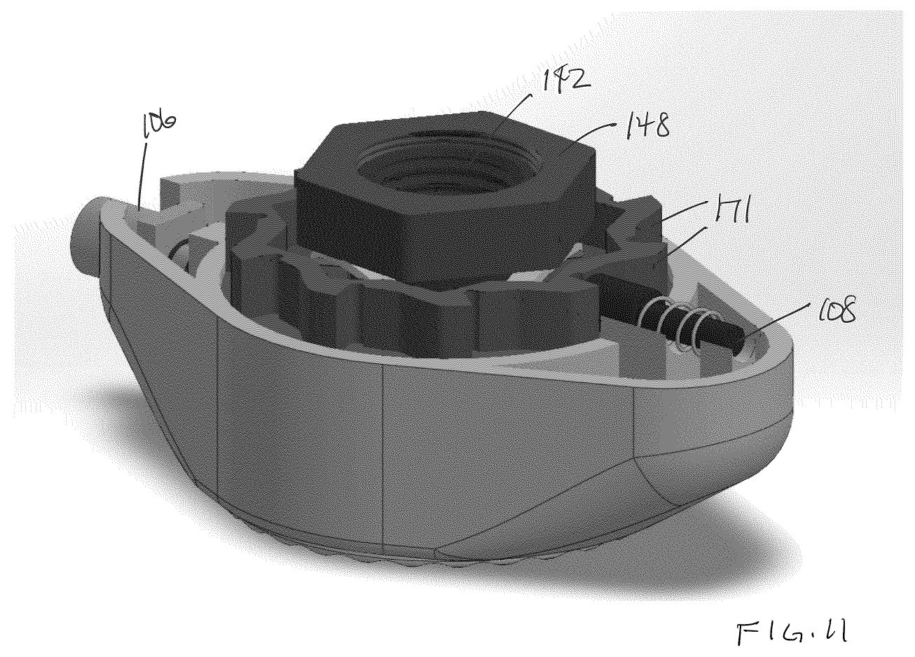

FIGS. 8-11, 12a-12f, and 13a-13c show the upper clamp housing 150 that includes most of the tightening and ratcheting mechanisms. As has already been described, and can be seen in the sequence from 12A to 12F, turning the upper clamp housing 150 in a clockwise direction turns the drive screw 140, which draws the central axle 126 upwards and closes the jaws 122, 124.

The way this works to close the jaws 122, 124 to a predefined pressure on the external fixation rod 300 is not through measuring pressure applied to the rod, or even measuring torque, but through the use of the gearing and wave spring washer 147.

When a surgeon initially clockwise-turns the upper clamp housing 150, the inwardly biased ratchet pin 108 engages outer drive washer teeth 171 on the outer drive washer 170. Turning the drive washer 170 turns the slip cam washer 160, which has slip cam washer teeth 161 engaged to the drive screw gearing 143, and thus also turns the drive screw 140, drawing the jaws 122, 124 closed via the upwards action of the central axle 126.

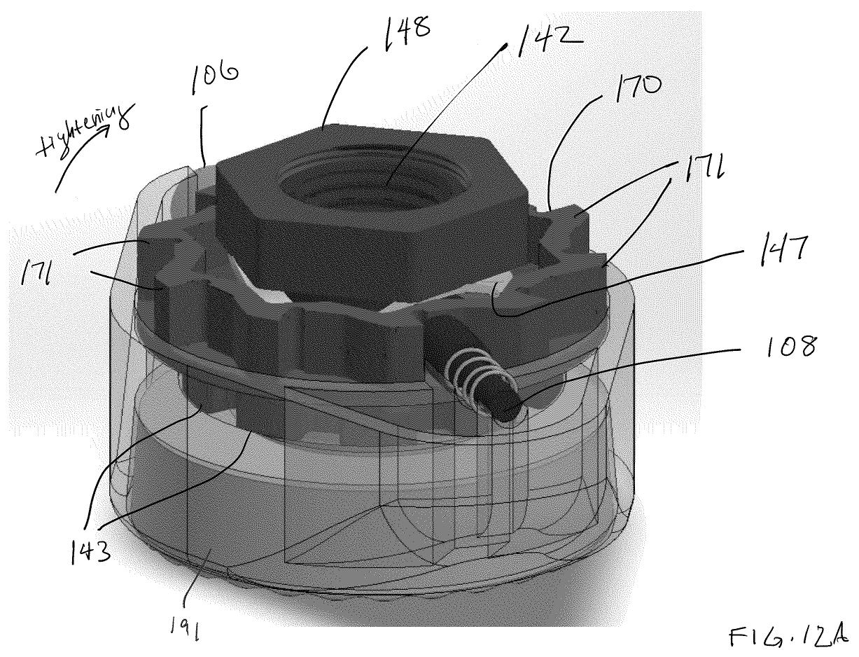

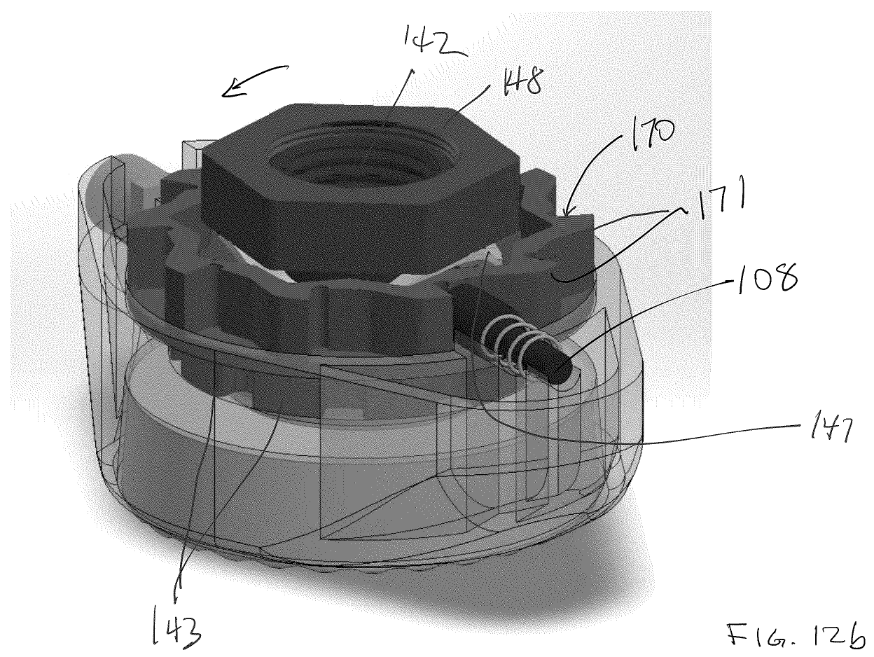

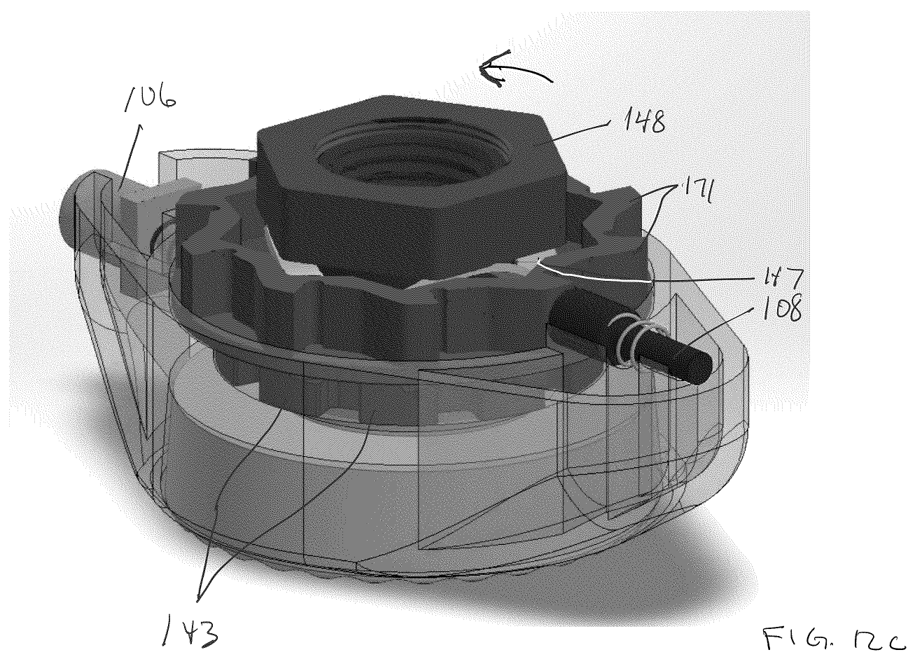

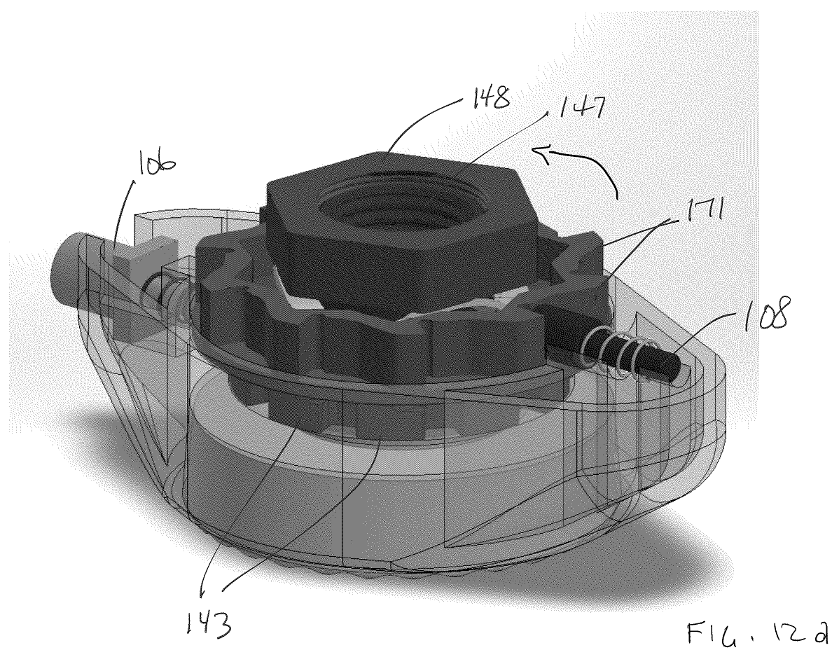

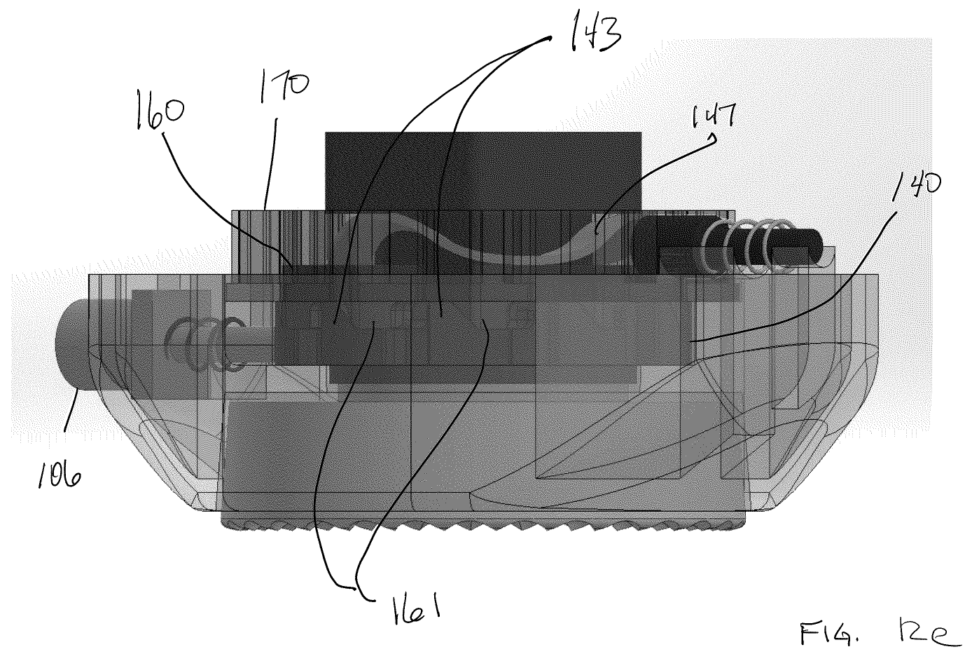

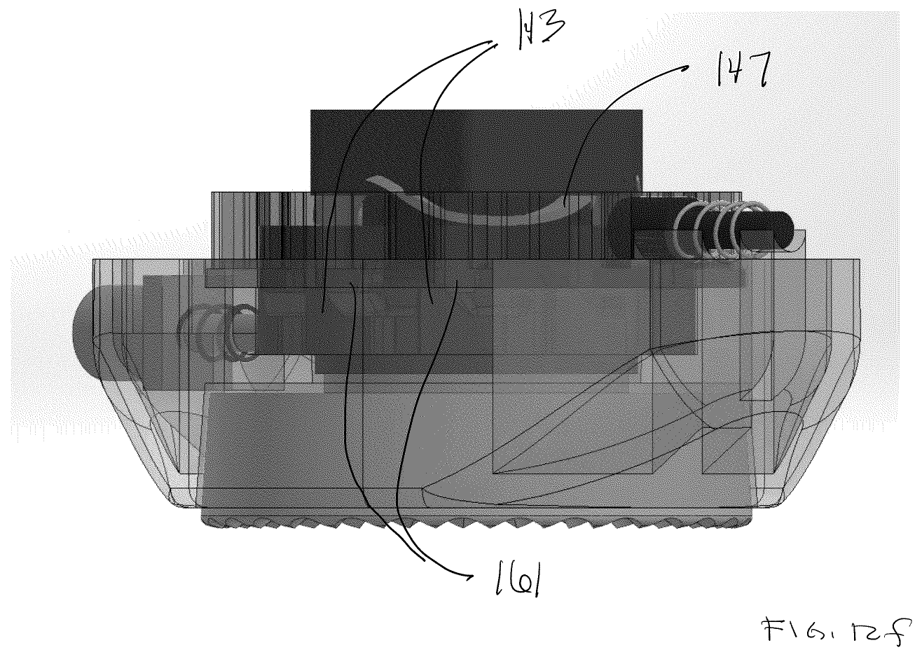

FIGS. 12b-12d show that after the surgeon achieves a certain engagement between the jaws 122, 124 and the external fixation rod 300, they can move the upper housing 150 in a counterclockwise motion, resulting in the ratchet pin 108 traveling up the ramp on the outer drive washer teeth 171, and dropping into the gap between the teeth for further tightening. This ratcheting movement would be familiar to people using ratcheting tools.

The engagement between the slip cam washer teeth 161 and the drive screw gearing 143 in FIGS. 12a to 12e is across a sloped surface, and the reason that the slip cam washer teeth 161 do not travel up and over the drive screw gears 143 along these sloped surfaces is because the wave spring washer 147 presses downwards against the slip cam washer 160, applying force to prevent the slip cam washer 160's upward travel.

As the jaws 122, 124 tighten on an external fixation rod 300, it becomes harder to draw the central axle 126 upwards via the drive screw 140's engagement therewith, and this results in a more upwards force being exerted by the slip cam washer 160 on the wave spring washer 147. FIG. 12f shows that as this force increases to a predetermined threshold, the slip cam washer 160 overcomes the downward force applied by the wave spring washer 147 and its teeth 160 ride up the ramp surface of the drive screw 143, resulting in no further turning of the drive screw 140, and thus, no further tightening of the jaws 122, 124 on the external fixation rod 300.

### Overtightening

If a surgeon is not satisfied with the tightening between the jaws 122, 124 and external fixation rod 300 achieved using the steps shown in FIGS. 12a-12f, they can overtighten beyond what the wave spring washer 147 would allow. To do this, the surgeon depresses the action pin 106, which drives the action pin 106 into engagement with the drive screw gears 143. Once this is done, the surgeon continues to turn the upper housing 150 in its clockwise manner. This movement turns the drive screw 140 (as well as the drive washer 170, slip washer 160, and wave spring washer 147) and further draws the central axle 126 upwards, closing the jaws 122, 124 further together around the external fixation rod 300.

### Releasing

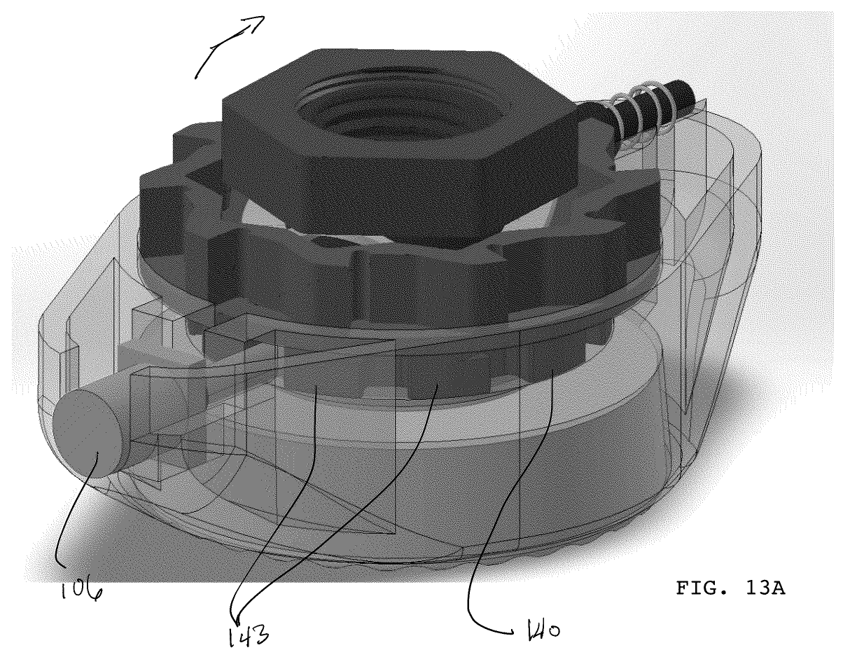

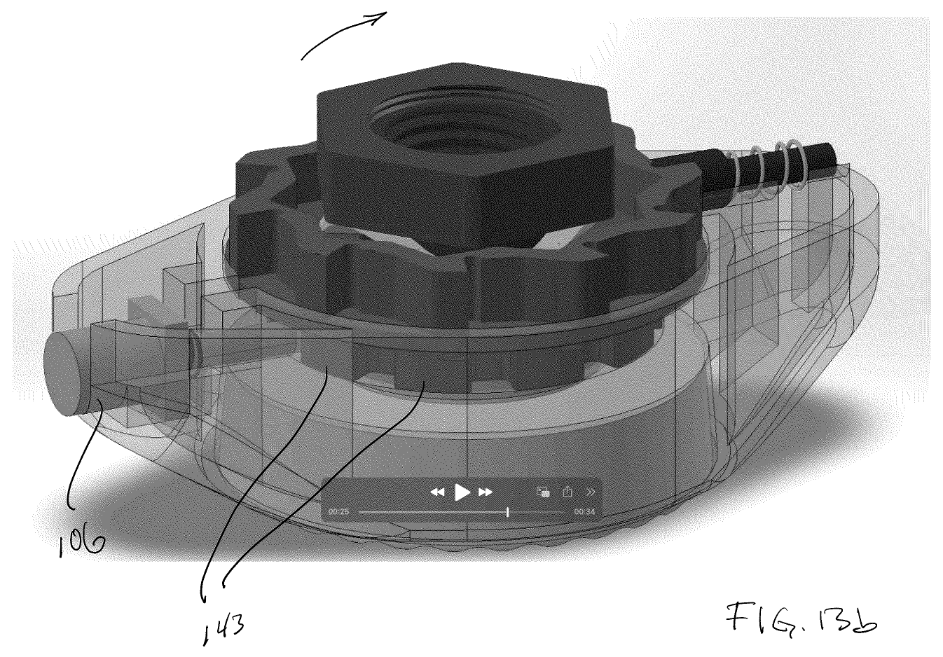

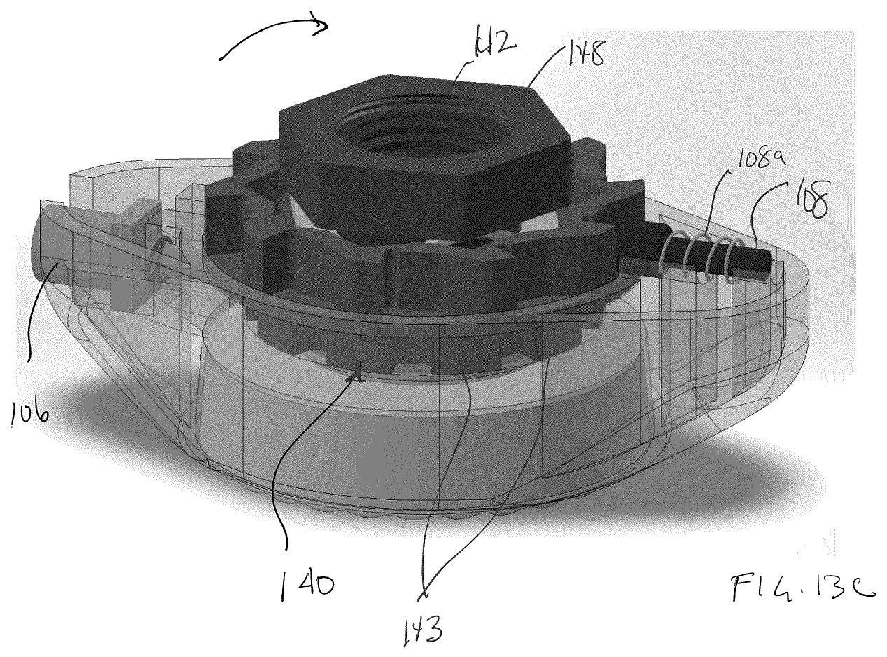

To release the jaws 122, 124 from around the external fixation rod 300, the steps in FIGS. 13a-c are played out in reverse, with the surgeon engaging the action pin 106, and turning the upper housing in a counter-clockwise motion, which drives the central axle 126 downwards, and releases the pressure of the jaws 122, 124 on the external fixation rod 300.

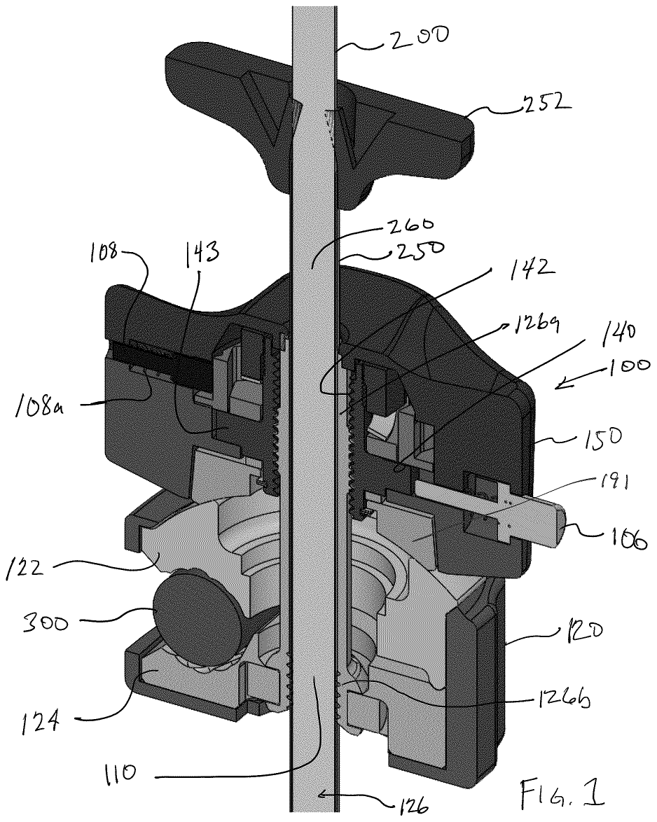

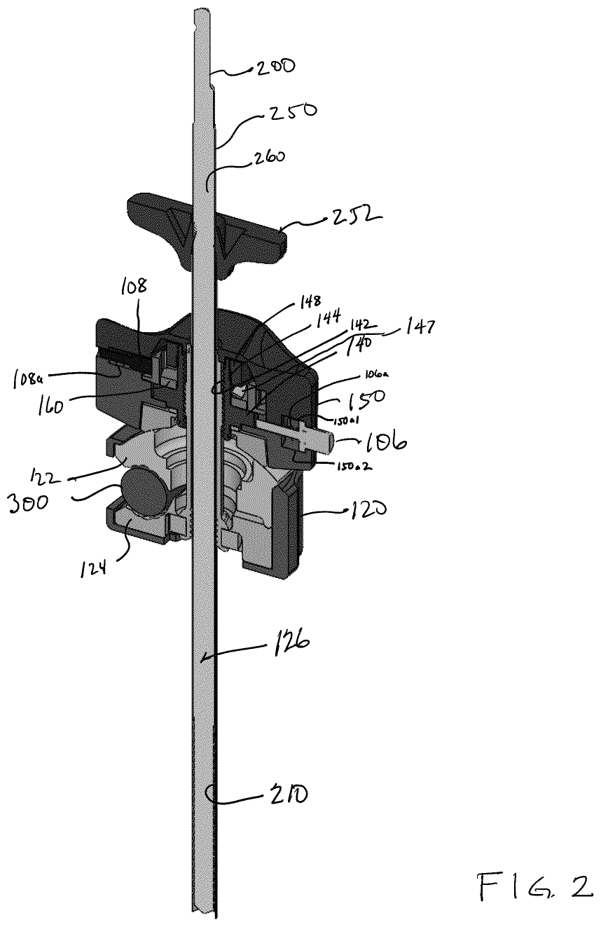

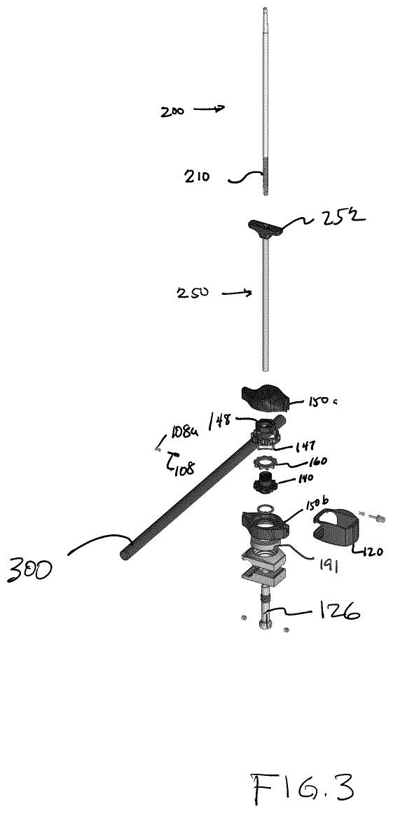

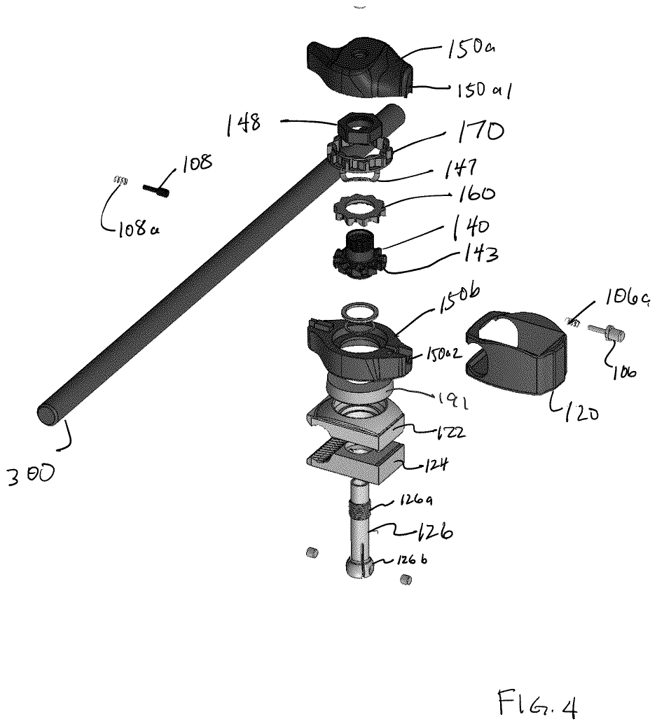

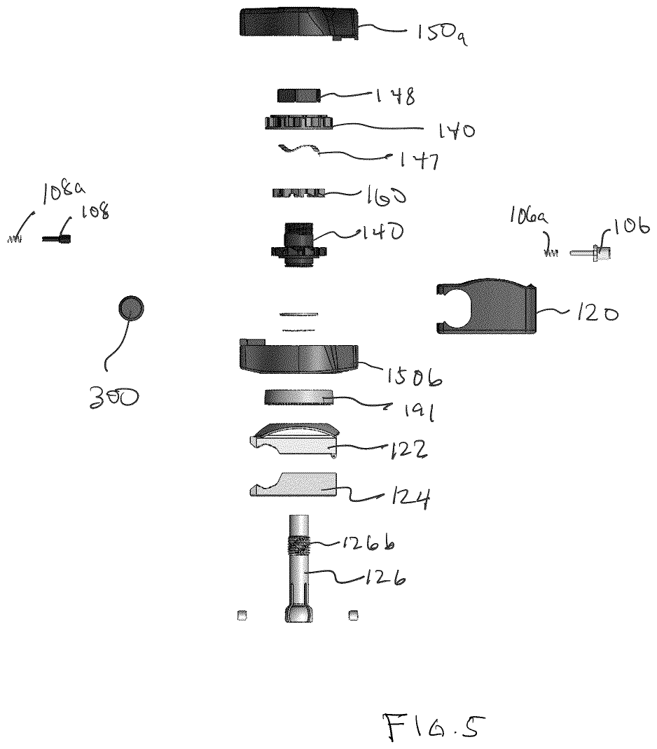

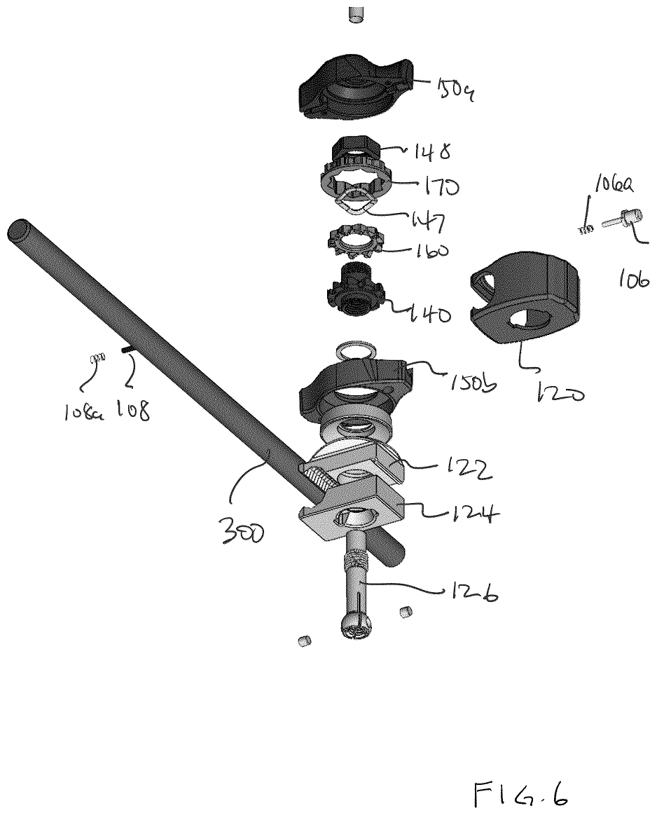

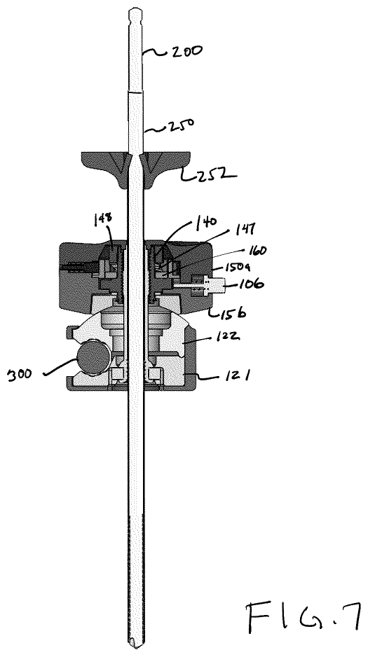

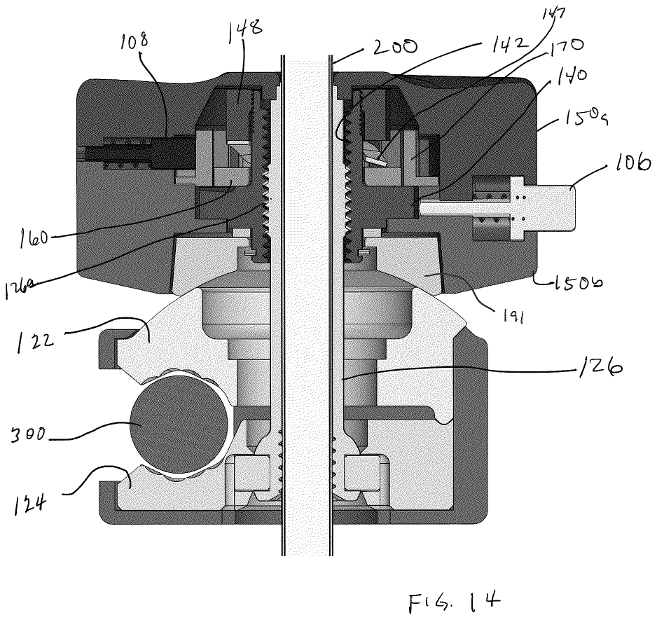

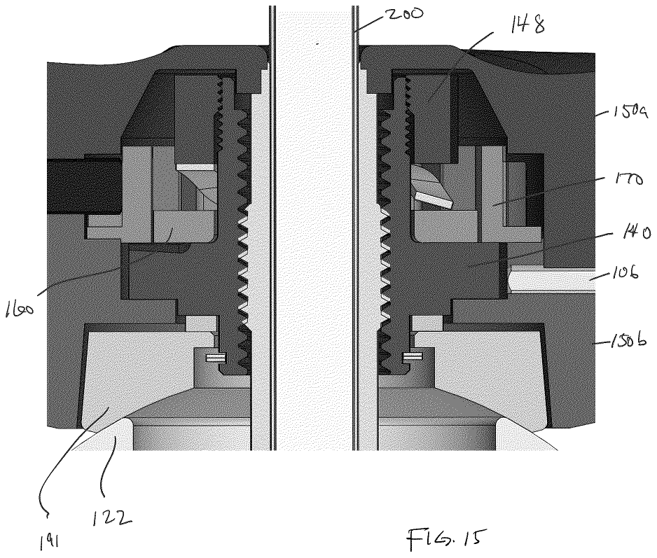

FIGS. 1-7, 14, and 15 show cross sections and exploded views of the clamping system to help further illustrate the parts used therein.

While the invention has been described with reference to the embodiments above, a person of ordinary skill in the art would understand that various changes or modifications may be made thereto without departing from the scope of the claims.

We claim:

1. A clamping device configured to engage an external fixation rod for use in bone fracture surgery comprising:
an upper housing including a ratcheting mechanism; and
a lower housing comprising an upper jaw and a lower jaw configured to engage an external fixation rod;
wherein when the upper housing is turned in a clockwise motion relative to the lower housing, the jaws are drawn towards one another until opposed by a predetermined force;
wherein engaging an action pin in the upper housing and turning the upper housing in a clockwise motion can overcome the predetermined force;
wherein engaging an action pin in the upper housing and turning the upper housing in a counterclockwise motion can release engagement to an external fixation rod.

2. The clamping device of claim 1, further comprising a wave spring washer that applies the predetermined force.

3. The clamping device of claim 2, further comprising a central axle engaged to the upper jaw and lower jaw, wherein the engagement between the central axle and the lower jaw is such that movement of the central axle moves the lower jaw.

4. The clamping device of claim 3, further comprising a drive screw engaged to the central axle through drive screw internal threading and central axel external threading, such that rotation of the drive screw draws the central axle upward or downward depending on clockwise or counterclockwise drive screw rotation, respectively.

5. The clamping device of claim 4, wherein a slip cam washer engages both the drive screw and the wave spring washer in such a way that when the predetermined force on the slip cam washer overcomes a downward force applied by the wave spring washer, slip cam gears on the slip cam washer slide over drive screw gears and clockwise motion of the upper housing will not further draw the jaws toward one another.

6. The clamping device of claim 5, wherein the action pin, when depressed, engages drive screw gears, and any rotation of the upper housing in a clockwise or counterclockwise motion draws the jaws towards or away from one another respectively.

7. The clamping device of clam 6, further comprising a ratchet pin that engages gears on an outer drive washer, wherein the outer drive washer is engaged to the slip cam washer, wherein counterclockwise rotation of the upper housing results in a ratchet action of the pin along outer drive washer gears that does not result in turning the drive screw or drawing the jaws toward one another.
